SEQUENCE LISTING (2 sequences)

SEQ ID NO: 1 - DNA, 37 bp, synthetic construct
  source 1..37: mol_type = other DNA
gttttcccagtcacgacgttgtaaaacgacggccagt

SEQ ID NO: 2 - DNA, 65 bp, synthetic construct
  source 1..65: mol_type = other DNA
ccccgggtaccgagctcgaattcactggccgtcgttttacaacgtcgtgactgggaaaaccctgg

(19) United States
(12) Patent Application Publication
(10) Pub. No.: US 2024/0401114 A1
(43) Pub. Date: Dec. 5, 2024

(54) HIGH SENSITIVITY AND RAPID NUCLEIC ACID DETECTION

(71) Applicant: GIT Applied Biotechnologies, LLC., East Windsor, NJ (US)

(72) Inventors: Guillermo TOUS, East Windsor, NJ (US); Cristina TOUS, East Windsor, NJ (US); Elena TOUS, Newtown, PA (US); Jose Ramon CASAS-FINET, Gaithersburg, MD (US)

(21) Appl. No.: 18/678,050

(22) Filed: May 30, 2024

Related U.S. Application Data
(60) Provisional application No. 63/504,890, filed on May 30, 2023.

(51) Int. Cl. C12Q 1/6825; C12Q 1/6804; C12Q 1/6806; C12Q 1/6844; C12Q 1/6876; G01N 27/327

(52) U.S. Cl. CPC C12Q 1/6825; C12Q 1/6804; C12Q 1/6806; C12Q 1/6844; C12Q 1/6876; G01N 27/3276

(57) ABSTRACT

The invention relates to novel methods and systems for detection and quantification of nucleic acids.

### RELATED APPLICATIONS AND INCORPORATION BY REFERENCE

This application claims priority to U.S. provisional application Ser. No. 63/504,890, filed May 30, 2023, which is incorporated by reference herein in its entirety.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy, was created Aug. 13, 2024, is named Y9598_01001.xml and is 2,963 bytes in size.

### FIELD OF THE INVENTION

The invention relates to novel methods and systems for detection and quantification of nucleic acids.

### BACKGROUND OF THE INVENTION

Several methods have been described in the literature and in patents for bacterial or viral clinical detection diagnostics (e.g. COVID-19 diagnostics), most of them involving PCR and/or ELISA based serological tests and UV or fluorescence detection.

These approaches have severe limitations: serological tests based on MABs and ELISAs depend on their MABs selectivity, avidity and at the end fluorescent signal amplification that it is background and matrix dependent.

PCR technology used to amplify trace amounts of DNA or RNA is ubiquitous, but has limitations, due principally to non-specific amplification (error rate), and the time it takes to achieve the desired amplification (e.g., twenty (20) to forty (40) cycles).

The invention provides methods and systems that offer improved detection and quantification of nucleic acids. Uses include clinical diagnostics and sequencing platforms. The methods offer rapid detection and identification of known and unknown pathogens, and biological threats.

Citation or identification of any document in this application is not an admission that such document is available as prior art to the present invention.

### SUMMARY OF THE INVENTION

The invention provides a method of detecting a target nucleic acid which comprises providing a first probe nucleic acid immobilized to the surface of a piezoelectric crystal or microbalance sensor, the first probe nucleic acid comprising a sequence complementary to the target nucleic acid; contacting the first probe nucleic acid with the target nucleic acid to form a nucleic acid hybrid; and detecting a change in mass of the sensor wherein a change in mass of the sensor indicates the presence of the target. In certain embodiments, the change in mass is quantified. In certain embodiments, the mass of the first probe-target nucleic acid hybrid is increased by the binding of a second probe nucleic acid which hybridizes to a different region of target nucleic acid, such as but not limited to a single-stranded DNA or RNA, optionally linked to mass element such as but not limited to a bead. In certain embodiments, the first probe nucleic acid and/or the target nucleic acid in the probe-target nucleic acid hybrid is elongated by a suitable polymerase or reverse transcriptase (RTP) and the mass of the elongated first probe-target nucleic acid hybrid is detected. In certain embodiments, the mass of the first probe-target nucleic acid hybrid is increased by the binding of a hybrid nucleic acid-specific antibody, optionally linked to a mass element such as but not limited to a bead. In certain embodiments, the mass of the first probe-target nucleic acid hybrid is increased by the binding of a hybrid nucleic acid-specific nucleic acid, such as but not limited to a single-stranded DNA or RNA, optionally linked to a mass element such as but not limited to a bead.

In an aspect, the invention provides a method for detecting a target nucleic acid in a test sample. In an embodiment, the method comprises (a) immobilizing a first probe nucleic acid on a solid support, (b) contacting the first probe nucleic acid with the target nucleic acid to form a probe-target complex, and (c) detecting hybridization of the first probe nucleic acid and the target nucleic acid by determining the mass of the immobilized probe-target complex.

In certain embodiments, the target nucleic acid in the sample comprises a viral nucleic acid. In certain embodiments, the target nucleic acid comprises a bacterial nucleic acid. In certain embodiments, the target nucleic acid comprises a microbial nucleic acid.

In certain embodiments, the target nucleic acid comprises a single-stranded nucleic acid. In certain embodiments, the target nucleic acid comprises a double-stranded nucleic acid. In certain embodiments, the target nucleic acid comprises RNA. In certain embodiments, the target nucleic acid comprises DNA.

In certain embodiments, the first probe nucleic acid comprises DNA. In certain embodiments, the first probe nucleic acid comprises RNA. In certain embodiments, the first probe comprises a single-stranded nucleic acid. In certain embodiments, the first probe comprises a double-stranded nucleic acid. In certain embodiments, the first probe nucleic acid comprises a sequence containing one or more non-natural nucleotides including, but not limited to, phosphodiester analogs, nucleobase analogs, and/or furanose ring substitutions.

In certain embodiments, the complex comprises a DNA-DNA duplex. In certain embodiments, the complex comprises a DNA-RNA duplex. In certain embodiments, the complex comprises an RNA-RNA duplex. In certain embodiments the complex comprises additional nucleic acids, which can be DNA, RNA, aptamer, or a combination thereof.

In certain embodiments, the method comprises an extraction whereby the target nucleic acid is released from an organism or particle, for example, without limitation, a cell, spore, or viral particle. In certain embodiments, the extraction is performed before the contacting step. In certain embodiments, the extraction is performed concurrently with the contacting step. In certain embodiments, extraction comprises treating a biological sample with a detergent such as SDS or an enzyme such trypsin or chymotrypsin or temperature or a combination thereof to obtain RNA or DNA.

In certain embodiments, the solid support comprises a piezoelectric material. In certain embodiments, the piezoelectric material comprises a quartz crystal. In certain embodiments, the solid support comprises a piezoelectric material coated with an electrode of gold, aluminum, silver, or an amalgam.

In certain embodiments, determining the mass change, i.e., the added mass of a complex comprising the first probe nucleic acid and the target nucleic acid, comprises measuring a change in the oscillation frequency of the piezoelectric material. In certain embodiments, determining the mass change comprises measuring a change in phase angle in the oscillations of the piezoelectric material. In certain embodiments, determining the mass change comprises measuring differential surface stress in a microcantilever sensor.

In another aspect, the invention provides a method for detecting a target nucleic acid in a test sample, wherein the method comprises: (a) immobilizing a first probe nucleic acid on a solid support, (b) contacting the first probe nucleic acid with the target nucleic acid to form a first probe-target complex, (c) contacting the first probe-target complex with a second probe nucleic acid which hybridizes to a different region of target nucleic acid, such as but not limited to a single-stranded DNA or RNA probe, optionally linked to or complexed with a mass element (i.e., a second probe nucleic acid complex) such as but not limited to a bead, and (d) detecting hybridization of the first probe nucleic acid with the target nucleic acid and second probe nucleic acid complex by determining the mass of the immobilized complex.

In certain embodiments, the target nucleic acid in the sample comprises a viral nucleic acid. In certain embodiments, the target nucleic acid comprises a bacterial nucleic acid. In certain embodiments, the target nucleic acid comprises a microbial nucleic acid.

In certain embodiments, the target nucleic acid comprises a single-stranded nucleic acid. In certain embodiments, the target nucleic acid comprises a double-stranded nucleic acid. In certain embodiments, the target nucleic acid comprises RNA. In certain embodiments, the target nucleic acid comprises DNA.

In certain embodiments, the probe nucleic acid comprises DNA. In certain embodiments, the probe nucleic acid comprises one or more non-natural nucleotides including, but not limited to, phosphodiester analogs, nucleobase analogs, and/or furanose ring substitutions. In certain embodiments, the probe nucleic acid comprises RNA. In certain embodiments, the probe comprises a single-stranded nucleic acid. In certain embodiments, the probe comprises a double-stranded nucleic acid.

In certain embodiments, the first probe-complex comprises a DNA-DNA duplex. In certain embodiments, the first probe complex comprises a DNA-RNA duplex. In certain embodiments, the first probe complex comprises an RNA-RNA duplex. In certain embodiments the first probe complex comprises additional nucleic acids, which can be DNA, RNA, aptamer, or combinations thereof.

In certain embodiments, the second probe nucleic acid that hybridizes to the first probe-complex is linked to a mass element. In certain embodiments, the mass element comprises a bead. In certain embodiments, the mass element comprises other enhancers such as, but not limited to, bioaffinity-labelled magnetic nanoparticles.

In certain embodiments, the method comprises an extraction whereby the target nucleic acid is released from an organism or particle, for example, without limitation, a cell, spore, or viral particle. In certain embodiments, the extraction is performed prior to the contacting step. In certain embodiments, the extraction is performed concurrently with the contacting step. In certain embodiments, extraction comprises treating a biological sample with a detergent such as SDS or an enzyme such trypsin or chymotrypsin or temperature or a combination thereof to obtain RNA or DNA.

In certain embodiments, the solid support comprises a piezoelectric material. In certain embodiments, the piezoelectric material comprises a quartz crystal. In certain embodiments, the solid support comprises a piezoelectric material coated with an electrode of gold, aluminum, silver, or an amalgam. In certain embodiments, a solid-mounted thin piezoelectric resonator is part of a microchip capable of additional or multiple sensing modes such as electrochemical, gravimetric and/or viscosimetric.

In certain embodiments, determining the mass change, i.e., the added mass of a complex or complexes comprising the first probe nucleic acid, the target nucleic acid and second probe nucleic acid or second probe nucleic acid complex, comprises measuring a change in the oscillation frequency of the piezoelectric material. In certain embodiments, determining the mass change comprises measuring a change in phase angle in the oscillations of the piezoelectric material.

In another aspect, the invention provides a method for detecting a target nucleic acid in a test sample, wherein the method comprises: (a) immobilizing a first probe nucleic acid on a solid support, (b) contacting the first probe nucleic acid with the target nucleic acid to form a first probe-target complex, (c) elongating one or more nucleic acids of the first probe-target complex, and (d) detecting hybridization of the probe nucleic acid and the target nucleic acid by determining the mass of the immobilized complex.

In certain embodiments, the target nucleic acid in the sample comprises a viral nucleic acid. In certain embodiments, the target nucleic acid comprises a bacterial nucleic acid. In certain embodiments, the target nucleic acid comprises a microbial nucleic acid.

In certain embodiments, the target nucleic acid comprises a single-stranded nucleic acid. In certain embodiments, the target nucleic acid comprises a double-stranded nucleic acid. In certain embodiments, the target nucleic acid comprises RNA. In certain embodiments, the target nucleic acid comprises DNA.

In certain embodiments, the first probe nucleic acid comprises DNA. In certain embodiments, the first probe nucleic acid comprises RNA. In certain embodiments, the first probe comprises a single-stranded nucleic acid. In certain embodiments, the first probe comprises a double-stranded nucleic acid. In certain embodiments, the first probe nucleic acid comprises one or more non-natural nucleotides including, but not limited to, phosphodiester analogs, nucleobase analogs, and/or furanose ring substitutions.

In certain embodiments, the complex comprises a DNA-DNA duplex. In certain embodiments, the complex comprises a DNA-RNA duplex. In certain embodiments, the complex comprises an RNA-RNA duplex. In certain embodiments the complex comprises additional nucleic acids, which can be DNA, RNA, aptamer, or combinations thereof.

In certain embodiments, the method comprises an extraction whereby the target nucleic acid is released from an organism or particle, for example, without limitation, a cell, spore, or viral particle. In certain embodiments, the extraction is performed prior to the contacting step. In certain embodiments, the extraction is performed concurrently with the contacting step. In certain embodiments, extraction comprises treating a biological sample with a detergent such as SDS or an enzyme such trypsin or chymotrypsin or temperature or a combination thereof to obtain RNA or DNA.

In certain embodiments, the solid support comprises a piezoelectric material. In certain embodiments, the piezoelectric material comprises a quartz crystal. In certain embodiments, the solid support comprises a piezoelectric material with an electrode of gold, aluminum, silver, or an amalgam. In certain embodiments, a solid-mounted thin piezoelectric resonator is part of a microchip capable of additional or multiple sensing modes.

In certain embodiments, determining the mass change, i.e., the added mass of a complex comprising the probe nucleic acid, the target nucleic acid, the elongated probe nucleic acid, comprises measuring a change in the oscillation frequency of the piezoelectric material. In certain embodiments, determining the mass change comprises measuring a change in phase angle in the oscillations of the piezoelectric material.

In another aspect, the invention provides a method for detecting a target nucleic acid in a test sample, wherein the method comprises: (a) immobilizing a probe nucleic acid on a solid support, (b) contacting the probe nucleic acid with the target nucleic acid to form a probe-target complex, (c) elongating one or more nucleic acids of the probe-target complex to form an elongated probe-target complex, (d) contacting the elongated probe-target complex with a binding protein specific for the elongated probe-target complex, and (e) detecting hybridization of the probe nucleic acid and the target nucleic acid by determining the mass of the immobilized complex.

In certain embodiments, the target nucleic acid in the sample comprises a viral nucleic acid. In certain embodiments, the target nucleic acid comprises a bacterial nucleic acid. In certain embodiments, the target nucleic acid comprises a microbial nucleic acid.

In certain embodiments, the target nucleic acid comprises a single-stranded nucleic acid. In certain embodiments, the target nucleic acid comprises a double-stranded nucleic acid. In certain embodiments, the target nucleic acid comprises RNA. In certain embodiments, the target nucleic acid comprises DNA.

In certain embodiments, the first probe nucleic acid comprises DNA. In certain embodiments, the first probe nucleic acid comprises RNA. In certain embodiments, the first probe comprises a single-stranded nucleic acid. In certain embodiments, the first probe comprises a double-stranded nucleic acid. In certain embodiments, the first probe nucleic acid comprises a sequence containing one or more non-natural nucleotides including, but not limited to, phosphodiester analogs, nucleobase analogs, and/or furanose ring substitutions.

In certain embodiments, the complex comprises a DNA-DNA duplex. In certain embodiments, the complex comprises a DNA-RNA duplex. In certain embodiments, the complex comprises an RNA-RNA duplex. In certain embodiments the complex comprises additional nucleic acids, which can be DNA, RNA, aptamer, or combinations thereof.

In certain embodiments, the binding protein specific for the elongated probe-target complex is an antibody or binding fragment thereof. In certain embodiments, the binding protein, antibody, or binding fragment thereof is linked to a mass element. In certain embodiments, the mass element comprises a bead.

In certain embodiments, the method comprises an extraction whereby the target nucleic acid is released from an organism or particle, for example, without limitation, a cell, spore, or viral particle. In certain embodiments, the extraction is performed prior to the contacting step. In certain embodiments, the extraction is performed concurrently with the contacting step. In certain embodiments, extraction comprises treating a biological sample with a detergent such as SDS or an enzyme such trypsin or chymotrypsin or temperature or a combination thereof to obtain RNA or DNA.

In certain embodiments, the solid support comprises a piezoelectric material. In certain embodiments, the piezoelectric material comprises a quartz crystal. In certain embodiments, the solid support comprises a piezoelectric material coated with an electrode of gold, aluminum, silver, or an amalgam. In certain embodiments, a solid-mounted thin piezoelectric resonator is part of a microchip capable of additional or multiple sensing modes.

In certain embodiments, determining the mass change, i.e., the added mass of a complex comprising the first probe nucleic acid, the target nucleic acid, the elongated probe nucleic acid, and the binding protein or i.e., MAB specific for the elongated hybrid probe-target complex comprises measuring a change in the oscillation frequency of the piezoelectric material. In certain embodiments, determining the mass change comprises measuring a change in phase angle in the oscillations of the piezoelectric material.

In another aspect, the invention provides a method for detecting a target nucleic acid in a test sample, wherein the method comprises: (a) immobilizing a first probe nucleic acid on a solid support, (b) contacting the first probe nucleic acid with the target nucleic acid to form a first probe-target complex, (c) elongating one or more nucleic acids of the first probe-target complex to form an elongated first probe-target complex, (d) contacting the elongated first probe-target complex with an indicator nucleic acid (e.g., a third probe nucleic acid) specific for the elongated first probe-target complex, and (e) detecting hybridization of the first probe nucleic acid, the target nucleic acid, elongated first probe nucleic acid, and complex indicator nucleic acid by determining the mass of the immobilized complex.

In certain embodiments, the target nucleic acid in the sample comprises a viral nucleic acid. In certain embodiments, the target nucleic acid comprises a bacterial nucleic acid. In certain embodiments, the target nucleic acid comprises a microbial nucleic acid.

In certain embodiments, the target nucleic acid comprises a single-stranded nucleic acid. In certain embodiments, the target nucleic acid comprises a double stranded nucleic acid. In certain embodiments, the target nucleic acid comprises RNA. In certain embodiments, the target nucleic acid comprises DNA.

In certain embodiments, the first probe nucleic acid comprises DNA. In certain embodiments, the first probe nucleic acid comprises RNA. In certain embodiments, the first probe comprises a single-stranded nucleic acid. In certain embodiments, the first probe comprises a double-stranded nucleic acid. In certain embodiments, the first probe nucleic acid comprises one or more non-natural nucleotides including, but not limited to, phosphodiester analogs, nucleobase analogs, and/or furanose ring substitutions.

In certain embodiments, the complex comprises a DNA-DNA duplex. In certain embodiments, the complex comprises a DNA-RNA duplex. In certain embodiments, the complex comprises an RNA-RNA duplex. In certain embodiments the complex comprises additional nucleic acids, which can be DNA, RNA, aptamer, or combinations thereof.

In certain embodiments the indicator nucleic acid (e.g., a third probe nucleic acid) comprises DNA. In certain embodiments, the indicator nucleic acid) comprises RNA. In certain embodiments the indicator nucleic acid is single-stranded. In certain embodiments, the indicator nucleic acid comprises two or three strands. In certain embodiments, the indicator nucleic acid is linked to a mass element. In certain embodiments, the mass element comprises a bead.

In certain embodiments, the method comprises an extraction whereby the target nucleic acid is released from an organism or particle, for example, without limitation, a cell, spore, or viral particle. In certain embodiments, the extraction is performed prior to the contacting step. In certain embodiments, the extraction is performed concurrently with the contacting step. In certain embodiments, extraction comprises treating a biological sample with a detergent such as SDS or an enzyme such trypsin or chymotrypsin or temperature or a combination thereof to obtain RNA or DNA.

In certain embodiments, the solid support comprises a piezoelectric material. In certain embodiments, the piezoelectric material comprises a quartz crystal. In certain embodiments, the solid support comprises a piezoelectric material coated with an electrode of gold, aluminum, silver, or an amalgam. In certain embodiments, a solid-mounted thin piezoelectric resonator is part of a microchip capable of additional or multiple sensing modes.

In certain embodiments, determining the mass change, i.e., the added mass of a complex comprising the first probe nucleic acid, the target nucleic acid, the elongated first probe nucleic acid, and complex indicator nucleic acid complex comprises measuring a change in the frequency of the piezoelectric material. In certain embodiments, determining the mass change comprises measuring a change in phase angle in the oscillations of the piezoelectric material. In certain embodiments, determining the mass change comprises measuring differential surfaces stress in a microcantilever sensor.

In another aspect, the invention provides a method for detecting a target nucleic acid in a test sample, wherein the method comprises: (a) immobilizing a first probe nucleic acid on a solid support, (b) contacting the first probe nucleic acid with the target nucleic acid to form a first probe nucleic acid-target complex, (c) elongating the first probe nucleic acid to form an elongated probe nucleic acid, (d) denaturing the elongated first probe nucleic acid-target complex, (e) contacting the elongated first probe with an indicator nucleic acid (e.g. a fourth probe nucleic acid) that binds specifically to the elongated first probe, and (e) detecting hybridization of the elongated first probe nucleic acid and the indicator nucleic acid (i.e. fourth probe nucleic acid) by determining the mass of the immobilized complex.

In certain embodiments, the target nucleic acid in the sample comprises a viral nucleic acid. In certain embodiments, the target nucleic acid comprises a bacterial nucleic acid. In certain embodiments, the target nucleic acid comprises a microbial nucleic acid.

In certain embodiments, the target nucleic acid comprises a single-stranded nucleic acid. In certain embodiments, the target nucleic acid comprises a double-stranded nucleic acid. In certain embodiments, the target nucleic acid comprises RNA. In certain embodiments, the target nucleic acid comprises DNA.

In certain embodiments, the first probe nucleic acid comprises DNA. In certain embodiments, the first probe nucleic acid comprises RNA. In certain embodiments, the first probe comprises a single-stranded nucleic acid. In certain embodiments, the first probe comprises a double-stranded nucleic acid. In certain embodiments, the first probe nucleic acid comprises one or more non-natural nucleotides including, but not limited to, phosphodiester analogs, nucleobase analogs, and/or furanose ring substitutions.

In certain embodiments, the complex comprises a DNA-DNA duplex. In certain embodiments, the complex comprises a DNA-RNA duplex. In certain embodiments, the complex comprises an RNA-RNA duplex. In certain embodiments the complex comprises additional nucleic acids, which can be DNA, RNA, aptamer, or combinations thereof.

In certain embodiments the indicator nucleic acid (or fourth probe nucleic acid) comprises DNA. In certain embodiments, the indicator nucleic acid comprises RNA. In certain embodiments the indicator nucleic acid is single stranded. In certain embodiments, the indicator nucleic acid comprises two or three strands. In certain embodiments, the indicator nucleic acid is linked to a mass element. In certain embodiments, the mass element comprises a bead.

In certain embodiments, the method comprises an extraction whereby the target nucleic acid is released from an organism or particle, for example, without limitation, a cell or viral particle. In certain embodiments, the extraction is performed prior to the contacting step. In certain embodiments, the extraction is performed concurrently with the contacting step. In certain embodiments, extraction comprises treating a biological sample with a detergent such as SDS or an enzyme such trypsin or chymotrypsin or temperature or a combination thereof to obtain RNA or DNA. In certain embodiments, a solid-mounted thin piezoelectric resonator is part of a microchip capable of additional or multiple sensing modes.

In certain embodiments, the solid support comprises a piezoelectric material. In certain embodiments, the piezoelectric material comprises a quartz crystal. In certain embodiments, the solid support comprises a piezoelectric material with an electrode of gold, aluminum, silver, or an amalgam.

In certain embodiments, determining the mass change, i.e., the added mass of a complex comprising the first probe nucleic acid, the target nucleic acid, elongated first probe nucleic acid and indicator nucleic acid (i.e. the fourth nucleic acid probe) comprises measuring a change in the frequency of the piezoelectric material. In certain embodiments, determining the mass change comprises measuring a change in phase angle in the oscillations of the piezoelectric material.

In another aspect, the invention provides a method for detecting a target nucleic acid in a test sample, wherein the method comprises: (a) immobilizing a first probe nucleic acid on a solid support, (b) contacting the first probe nucleic acid with the target nucleic acid to form a probe-target complex, (c) elongating the first probe nucleic acid to form an elongated first probe nucleic acid, (d) denaturing the elongated first probe-target complex, (e) contacting the elongated first probe with a primer nucleic acid (also referred to as a fifth nucleic acid probe) complementary to the elongated first probe at or near the 3′ end of the elongated probe, (f) extending the primer nucleic acid along the elongated first probe to produce a replicant target nucleic acid comprising all or part of the target nucleic acid, and (g) detecting hybridization of elongated first probe nucleic acids to target nucleic acid and replicant target nucleic acid by determining the mass of the immobilized complex.

In certain embodiments, steps (d), (e), and (f) are repeated one or more times.

In certain embodiments, the target nucleic acid in the sample comprises a viral nucleic acid. In certain embodiments, the target nucleic acid comprises a bacterial nucleic acid. In certain embodiments, the target nucleic acid comprises a microbial nucleic acid.

In certain embodiments, the target nucleic acid comprises a single-stranded nucleic acid. In certain embodiments, the target nucleic acid comprises a double-stranded nucleic acid. In certain embodiments, the target nucleic acid comprises RNA. In certain embodiments, the target nucleic acid comprises DNA.

In certain embodiments, the first probe nucleic acid comprises DNA. In certain embodiments, the first probe nucleic acid comprises RNA. In certain embodiments, the first probe comprises a single-stranded nucleic acid. In certain embodiments, the first probe comprises a double-stranded nucleic acid. In certain embodiments, the first probe nucleic acid comprises one or more non-natural nucleotides including, but not limited to, phosphodiester analogs, nucleobase analogs, and/or furanose ring substitutions.

In certain embodiments, the complex comprises a DNA-DNA duplex. In certain embodiments, the complex comprises a DNA-RNA duplex. In certain embodiments, the complex comprises an RNA-RNA duplex. In certain embodiments the complex comprises additional nucleic acids, which can be DNA, RNA, aptamer, or combinations thereof.

In certain embodiments, the method comprises an extraction whereby the target nucleic acid is released from an organism or particle, for example, without limitation, a cell or viral particle. In certain embodiments, the extraction is performed prior to the contacting step. In certain embodiments, the extraction is performed concurrently with the contacting step. In certain embodiments, extraction comprises treating a biological sample with a detergent such as SDS or an enzyme such trypsin or chymotrypsin or temperature or a combination thereof to obtain RNA or DNA.

In certain embodiments, the solid support comprises a piezoelectric material. In certain embodiments, the piezoelectric material comprises a quartz crystal. In certain embodiments, the solid support comprises a piezoelectric material with an electrode of gold, aluminum, silver, or an amalgam. In certain embodiments, a solid-mounted thin film piezoelectric resonator is part of a microchip capable of additional or multiple sensing modes.

In certain embodiments, determining the mass change, i.e., the added mass of a complex comprising the first probe nucleic acid, the target nucleic acid, elongated first probe nucleic acid, extended primer nucleic acid and any additional complex component comprises measuring a change in the frequency of the piezoelectric material. In certain embodiments, determining the mass change comprises measuring a change in phase angle in the oscillations of the piezoelectric material.

In another aspect, the invention provides a system for identifying a target nucleic acid in a sample comprising (a) a surface capable of functioning as a transducer, the surface having bound thereto a probe nucleic acid complementary at the 3′ end to a segment of the target nucleic acid; and (b) a polymerase capable of target nucleic acid-dependent addition of one or more nucleotides to the 3′ end of the probe nucleic acid to form an extended probe nucleic acid.

In certain embodiments, the sensor comprises a piezoelectric sensor. In certain embodiments, the sensor comprises a quartz crystal and an electrode comprising gold, silver, aluminum, or an amalgam.

In certain embodiments, the system comprises a molecular probe that specifically binds to a hybrid nucleic acid comprising the target nucleic acid and the extended first probe nucleic acid. In certain embodiments, the molecular probe comprises an antibody or binding fragment thereof. In certain embodiments, the molecular probe comprises or is linked to a mass element. In certain embodiments, the molecular probe comprises a nucleic acid. In certain embodiments, the molecular probe comprises a nucleic acid linked to a mass element.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises,” “comprised,” “comprising” and the like can have the meaning attributed to it in U.S. Patent law; e.g., they can mean “includes,” “included,” “including,” and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 depicts a quartz crystal microbalance (QCM) for detecting a target nucleic acid. QCMs are typically fabricated of a thin plate of quartz with affixed electrodes of gold, platinum, or silver. The QCM is adapted to display a probe nucleic acid that hybridizes specifically to a target.

FIG. 2 depicts signal amplification by three different approaches or mechanisms. 2a) Signal amplification by elongation by reverse transcriptase (RTP) or other suitable polymerase of nucleic acid bound first probe to piezoelectric crystal. 2b) Signal amplification by binding of second probe nucleic acid derivatized beads or complex of known mass to form a sub-picogram or picogram binding adduct to target nucleic acid that was previously captured by nucleic acid first probe bound to piezoelectric crystal. 3b) Signal amplification by binding of MAB derivatized beads or complex of known mass to form a sub-picogram or picogram binding adduct to target nucleic acid that was previously captured by first probe nucleic acid bound to piezoelectric crystal and elongated to form a hybrid DNA/RNA molecular structure. Mass-amplified RNA or DNA hybrids are detected with a piezoelectric crystal.

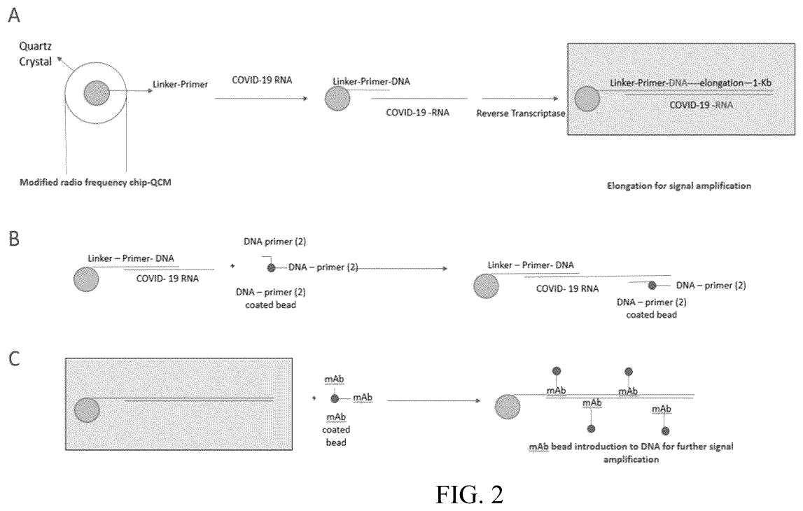

FIG. 3 depicts three different mechanisms to derivatize the piezoelectric crystal: 1) mercury (Hg) addition to gold center wafer followed with direct thio-linked probe reaction, 2) Hg addition to gold wafer followed by linker extension with maleimido group and reaction with thio-linked probe, 3) Hg addition to gold wafer followed by linker extension with thio-linked-NHS and reaction with amino-linked probe.

### DETAILED DESCRIPTION OF THE INVENTION

### Assay Technology

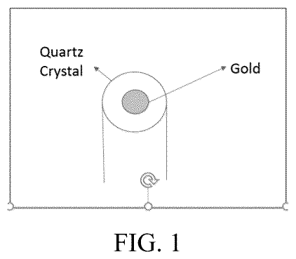

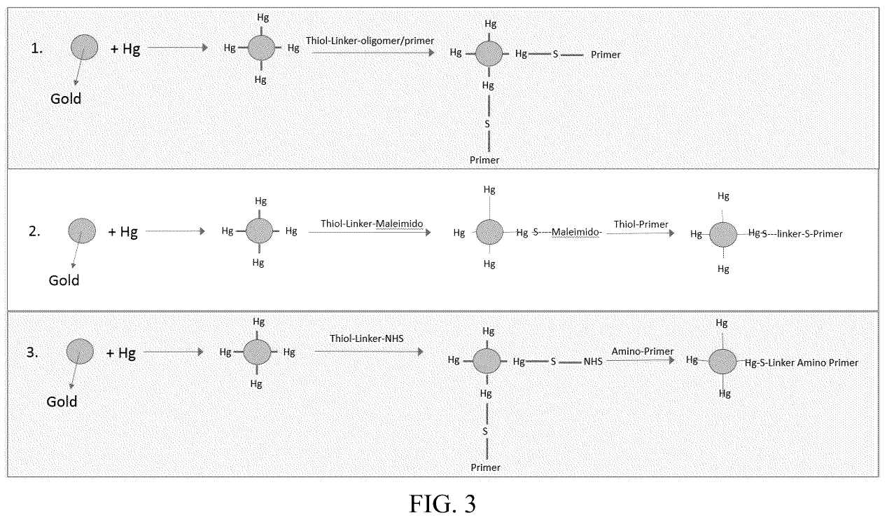

The method of the invention involves some or all of the following steps: sample processing (e.g. lysis) to release nucleic acid components, nucleic acid capture of target nucleic acids from the sample by hybridization with a probe (e.g. a first nucleic acid probe), optional wash to remove unbound components and debris, and elongation of one or both nucleic acids of the hybrid. In certain embodiments, the first probe nucleic acid comprises a linked amino primer. In certain embodiments, the first nucleic acid probe is immobilized to a detection system via reaction with the linked amino primer (e.g., FIG. 3). In certain embodiments, the detection system comprises a piezoelectric crystal (FIG. 1). In certain embodiments, it is sufficient to elongate the immobilized probe nucleic acid, for example by polymerase or reverse transcription. This method provides for signal amplification through increasing adduct mass via one or more of the following mechanisms.
1. Elongation of the DNA probe attached to the piezoelectric crystal along the captured RNA chain (reverse transcriptase polymerase; RTP elongation) to form an extended hybrid DNA/RNA duplex. The mass on the piezoelectric crystal is increased, hence there is a larger shift of the resonance energy.
2. Binding of a second probe nucleic acid specific against a different region of target nucleic acid, optionally linked to mass element such as but not limited to a bead (a second probe nucleic acid complex). Binding of the second probe nucleic acid to the target RNA or DNA increases the mass on the piezoelectric crystal, hence a larger shift of resonance energy.
3. Binding of a DNA/RNA hybrid-specific antibody (e.g., monoclonal antibody-S9.6). Binding of the antibody to the DNA/RNA heteroduplex increases the mass on the piezoelectric crystal, hence a larger shift of resonance energy.
4. Binding of a DNA/RNA hybrid-specific MAB-coated bead of a determined weight. Binding of a DNA/RNA hybrid-specific antibody coated bead increases the mass on the piezoelectric crystal, hence a larger shift of resonance energy.
5. Binding of a molecular probe specific for the probe-target DNA/RNA hybrid, such as but not limited to a nucleic acid specific for the probe-target DNA/RNA hybrid to form a triple helix. The molecular probe (e.g., the third nucleic acid strand) is optionally coupled to a mass element, such as but not limited to a bead. Binding of the DNA/RNA hybrid-specific molecular probe increases the mass on the piezoelectric crystal, hence a larger shift of resonance energy.
6. Synergistic combination of elongation and PCR amplification (RTP elongation followed by PCR) using reverse transcriptase and followed as in 1, 3, 4 or 5 above. In certain embodiments, the combination comprises two or more of:
a. Using an antibody with DNA/RNA hybrid affinity: increase of mass on the piezoelectric crystal as per 2 above, hence a larger shift of resonance energy.
b. Using an antibody with DNA/RNA hybrid affinity attached to a bead of a determined weight, to increase of mass on the piezoelectric crystal, hence a larger shift of resonance energy.
c. Using a molecular probe attached to a bead of a determined weight with affinity to the heteroduplex duplex or hybrid DNA/RNA complex to form a triple helix (R configuration), with increased mass on the piezoelectric crystal by the triplex structure once formed, hence a larger shift of resonance energy.
d. Using a molecular probe attached to a bead of a determined weight with affinity to elongated DNA molecule from viral RNA attached to piezoelectric crystal. First the elongated DNA/RNA hybrid duplex is denatured by raising the temperature, and the elongated first probe nucleic acid attached to the piezoelectric crystal is then bound to a probe-linked bead of known weight, to increase of mass attached on the piezoelectric crystal, hence larger shift of resonance energy.
e. Using a molecular probe attached to a bead of a determined weight with affinity to target RNA-molecule from viral RNA to increase of mass on the piezoelectric crystal, hence larger shift of resonance energy.

Signal amplification will be correlated with increased mass of the elongated DNA/RNA duplex, the elongated DNA duplex or RNA duplex, captured ss-DNA or captured ss-RNA and molecular probe-bead complex, the hybrid DNA/RNA MAB complex, the hybrid DNA/RNA MAB-bead complex, or the hybrid DNA/RNA molecular probe-bead complex.

Piezoelectric crystal resonance sensitivity can detect sub-picogram amounts, hence if the MAB-bead has a 0.1 picogram to 1 picogram weight, the piezoelectric crystal will detect one (1) RNA strand, since multiple beads are expected to bind the DNA/RNA hybrid complex. Current piezoelectric crystals can at least detect a weight shift of 1-20 picograms. A genome size of a virus ranges from 2 kilobases (kb) to 1 megabases (mb). One megabase is equal to 1,000,000 bases and it is equal to 0.001 picograms in mass. One thousand “mb” (1,000 mb) is equal to 1 picogram (10 exp (−12) grams) or approximately 1 billion base pairs. This is called a “C” value and it is the mass of DNA in pg (1 pg=approximately 1 billion base pairs of DNA (See, S.K. Sessions-Brenner's Encyclopedia of Genetics, second edition, 2013) in a haploid set of chromosomes.

Further sensitivity may be achieved through use of PCR with target-specific forward and reverse primers and probes. Greater than 100-fold amplification can be achieved in seven PCR cycles with limited increase in noise, and a 1,000-fold amplification can be achieved with ten PCR cycles.

### Amplification

In certain aspects, the invention includes linear amplification, exponential amplification, or both in order to benefit from complementary advantages. Following target-probe hybridization and extension of the probe nucleic acid, the extended probe-target hybrid is denatured and the extended probe hybridized to a primer complementary to the extended probe, preferably at or near the 3′ end. The primer is then extended with a polymerase or a reverse transcriptase (RT) to generate a new strand identical in sequence to the target and the process is repeated. In certain embodiments the amplification is linear, relying on a single primer to generate copies of the target. In certain embodiments the amplification is exponential, using a primer complementary to the 3′ end of the extended probe nucleic acid and a primer complementary to the 3′ end of the target nucleic acid. Linear amplification has the benefit of generating only strands complementary to the probe nucleic acid without copies of the probe nucleic acid strand that can hybridize with the target and be washed away. Exponential amplification has the benefit of fewer cycles required to generate the same number of target nucleic acids. In certain embodiments, amplification is asymmetric, using a higher amount of the primer complementary to the probe nucleic acid and a lower amount of the primer complementary to the target nucleic acid. The asymmetric amplification is initially exponential and becomes linear as the lesser primer is exhausted first. In an asymmetric amplification, the primers can be in ratios of 2:1 to 500:1, including 5:1, 10:1, 20:1, 50:1, 100:1, and 200:1. A Linear-After-The-Exponential-PCR-(Late-PCR) protocol, which uses a limiting primer with a higher melting temperature than the excess primer can be used to maintain reaction efficiency as the limiting primer concentration decreases mid-reaction.

### Sensor

The piezoelectric effect describes the relationship between a mechanical stress and an electric voltage in solids such as quartz and barium titanate (BaTiO3)) that exhibit this effect. When a mechanical stress is applied to these materials, they generate a voltage. The effect is reversible as well. When a voltage is applied to the material, the shape of the material changes.

Non-limiting examples of piezoelectric materials of the invention include, crystals such as gallium orthophosphate (GaPO₄) and langasite (La₃Ga₅SiO₁₄) (both quartz analogs), piezoelectric ceramics such as barium titanate (BaTiO₃), lead titanate (PbTiO₃), lead zirconate titanate (PZT), potassium niobate (KNbO₃), lithium niobate (LiNbO₃), lithium tantalate (LiTaO₃), sodium tungstate (Na₂WO₄), as well as lead-free sodium potassium niobate (NaKNb), bismuth ferrite (BiFeO₃), and sodium niobate (NaNbO₃), and organic piezoelectric materials such as poly(vinylidene fluoride) (PVDF) and its copolymers.

Useful electrodes include, without limitation, electrodes comprising gold, silver, aluminum, zinc, nickel, or amalgams.

### Molecular Probe

In certain embodiments, the invention includes a molecular probe that specifically binds to target nucleic acid captured by primer-1 or a probe nucleic acid-target nucleic acid hybrid or duplex. In certain embodiments, the molecular probe comprises an antibody or binding fragment thereof which binds to the probe nucleic acid-target nucleic acid hybrid or duplex. In certain embodiments, the molecular probe comprises a nucleic acid which binds to the probe nucleic acid-target nucleic acid hybrid or duplex.

In certain embodiment, the invention includes a molecular probe that specifically binds to nucleic acid duplex structures, i.e. RNA: DNA hybrid structures, through the use of a specific antibody or to double-stranded DNA through analog methods.

Design of the nucleic acid molecular probes is a feature of biosensor design, often determining its detection limit and analytical performance. Triplex nucleic acid nanostructures such as those formed between a DNA duplex and single-stranded RNA, or assemblies in which all three strands are DNA, present specific design challenges. Nevertheless, triplex nucleic acid complexes suitable for detection can be generated by extension of a nucleic acid, such as a first probe nucleic acid, by polymerization or reverse transcription along a target or other template, and triplex formation with a designed molecular beacon. For example, Li, Y. et al., 2015, Sci. Rep. 5 (1), 13010 describes a thrombin controlled amplification system to create a substrate for triplex formation. In certain embodiments of the invention, probe nucleic acid-target nucleic acid structures are extended to produce nucleic acid substrates for triplex formation.

Although the present invention and its advantages have been described in detail, it should be understood that various changes, substitutions, and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

Fabrication of a gold-coated piezoelectrically-excited sensing surface.

Specific adsorption of analytes of interest onto the formed layer can be continuously measured through a resonant frequency decrease stemming upon mass increase on a self-assembled monolayer, allowing direct quantification of the adsorbed amount.
(1) A piezoelectric crystal is first cleaned with acetone ((CH₃)₂CO) to remove excess oil, followed by water. The entire crystal is then dipped in liquid mercury metal, in order to prepare an activated gold surface that will form an amalgam useful in the quartz crystal liquid phase biosensor. The piezoelectric crystal is then shaken and dried to remove the excess Hg, after which it is placed in a solution containing (10%-50%) of nitric acid (HNO₃) overnight. The crystal is then removed and washed with pure water to remove excess acid. It is recommended to use an ultrasonic bath for washing the surfaces of the crystal. A useful washing sequence involves the use of water, EDTA (to remove any excess mercury ions that may remain), NaOH, and finally water. The crystal is allowed to dry, after which it is ready to be used as a quartz crystal liquid phase biosensor.
(2) The steps of procedure 1 are followed, except that the crystal is first cleaned, and then dipped in a solution of hydrochloric acid (HCl) before the amalgam is formed. The presence of chloride ion (Cl) results in the formation of a stronger bonding of the biological molecules retained on the activated gold surface thus formed by suppressing the disproportionation of Au (I) and improving its surface properties and stability (ref. 9).
(3) The steps of procedure 1 are followed, except that the oxidizing agent employed for the dissolution of the amalgam is HCl combined with hydrogen peroxide (H₂O₂). Alternatively, a strong oxidizing acid such as perchloric acid (HClO₄) may be substituted for the mixture of HCl and H₂O₂.

### Example 2

Loading of Cystine to Gold Foil as Surrogate Process for Preparing an Activated Gold Piezoelectric Crystal

Evaluation of the activation of gold crystal surface was performed by the addition of radioactive cystine (Cyst*), having the molecular formula: HO₂C*(NH₂)—C*H—C*H₂—S—S—C*H₂—C*H—(NH₂) C*O₂H, where (*) indicated a radioactive ¹⁴C₁₂ atom.

The reaction on gold foil was performed as follows: a solution containing 650 pmol of Cyst* was prepared in 200 μL of PBS (phosphate buffered saline) in a 500-μL polypropylene vial, and then the activated gold foil (1-inch long, 0.025-inch wide, 0.015-inch thick, 16.13 mm² one-sided area) was introduced.

Cystine molecules were adsorbed on the gold foil proportionally to the activated gold surface. The amount of radioactive cystine adsorbed on the gold surface was measured directly over time by subtraction using a liquid-scintillation counter, and by direct release of attached cystine to the gold foil.

Release of attached cystine was performed as follows: the gold foil was removed from the vial, washed with water to remove excess PBS and Cyst*, and then placed in a solution containing DTT (dithiothreitol: HS—CH(OH)—CH(OH)—CH(OH)—SH). DTT reduces the disulfide linkage of Cyst* within minutes, hence releasing cysteine, and allowing release of cysteine determination from gold foil.

### Example 3

### Piezoelectric Crystal Quartz Activation by Si—OH

Silica pre-activation—To activate the Si—OH portion of the gold piezoelectric crystal, several entire crystals are dipped into 10 mL of 10% glacial acetic acid in water at room temperature for 2-3 hours. The solution is decanted, and the piezoelectric crystals are washed 3 times with 10 mL of methanol (CH₃OH) and then ether, followed by additional drying under vacuum for 2 hours. The pre-activated quartz crystals are then crosslinked with 3-aminopropyl trimethoxy silane (APTMS, Sigma Chemical Co., St. Louis, MO).

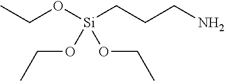

### Crosslinking is Carried Out Following the Procedure Below:

The pre-activated Si—OH surface is dipped into 10 mL of tetrachloromethane (CCl₄), and 500 μL of APTMS (H₂N—(CH₂)₃—Si—(OCH₃)₃) is added. The reaction is allowed to proceed for at least 3 hours (up to overnight) at room temperature, while swirling or going end-over-end (depending on the desired loading). After the reaction is complete, the piezoelectric crystals are removed and washed 4 times with 10 mL of CCl4 to remove the excess reagent. The amino-linked silica is then crosslinked with ethylene glycol-bis(succinic acid-N-hydroxysuccinimide ester) (EGS, chemical structure shown below) or sulfo-EGS (CovaChem, LLC., Loves Park, IL), depending on reaction conditions.

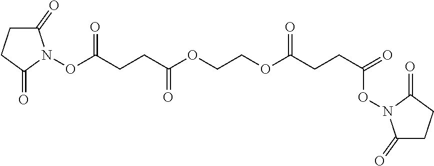 [SMILES: O=C(CCC(=O)ON1C(=O)CCC1=O)OCCOC(=O)CCC(=O)ON1C(=O)CCC1=O]

To this end, several pre-activated piezoelectric crystals are dipped into 10 mL of 10 mM NaH₂PO₄ buffer, pH 7.3. Then, 50 mg of EGS is added to 400 μL of dimethylformamide (DMF, (CH3)2NC(O) H), the pH is adjusted to 7.4 with 1 M Na₂HPO₄, pH 9.0, and the reaction is allowed to proceed for 45 minutes. The piezoelectric crystals are then removed from the reaction vial, placed on a Whatman filter #2 (Whatman Scientific Co., Maidstone, United Kingdom), and washed six times with 30 mL of water.

### Example 4

### Synthesis of 38-Mer Oligonucleotide Probe

A 38-mer nucleotide probe was synthesized on a Biosearch Cyclone 8400 Plus DNA synthesizer (Milligen/Biosearch, Ventura, CA), Trityl Group-On, at the 1 μM synthesis scale using a T ABI column (ThermoFisher Scientific, Waltham, MA). The synthesis was repeated three times; in each instance, an orange coloration representative of a trityl group was observed. Trityl content was not directly assessed.

Once the initial 37-mer (see below) was synthesized, a bottle of monomethoxy-trityl (MMT) B-(link)-beta-cyanoethyl phosphoramidite was placed in position number six. The link was then diluted with anhydrous acetonitrile, and the initial 37-mer reacted with the amino-link to yield the desired 38-mer below, after deprotection.

Oligonucleotide probe:
(SEQ ID NO: 1)
3′-TGA CCG GCA GCA AAA TGT TGC AGC ACT GAC
CCT TTT G-NH₂-′5

Amino-Link: MMT-NH—(CH₂)₆—O—P—(OCNEt)N-(iPr)₂

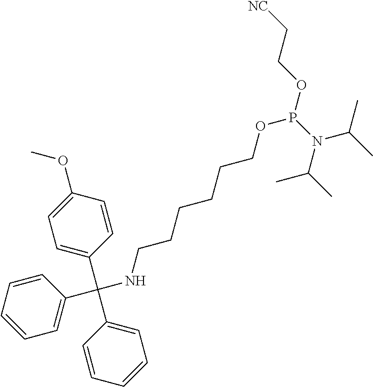 [SMILES: COc1ccc(C(NCCCCCCOP(OCCC#N)N(C(C)C)C(C)C)(c2ccccc2)c2ccccc2)cc1]

Monomethoxy-trityl (MMT) B-(link)-beta-cyanoethyl phosphoramidite linker attachment to 37-mer and cleavage of MMT group

150 mg of the linker compound was mixed with 4 mL of amidite diluent (Dry Diluent, Biosearch Technologies, Hoddesdon, United Kingdom); the linker compound is a viscous liquid and requires time to dissolve. The solution is transferred to the “X” reservoir of the Cyclone DNA synthesizer (Milligen/Biosearch, Ventura, CA) and the sequence of the desired oligonucleotide (see above) is entered, with an “X” for the 5′ terminal base (38-residue-“NH₂”). The final DMT is not removed prior to proceeding to cleavage of the monomethoxytrityl (MMT) group.

The MMT group is cleaved by disconnecting the T column from the Cyclone instrument at both ends, then connecting two 5-mL syringes at either end of the T column with a Luer-lock coupling (McMaster-Carr, Robbinsville, NJ). The first syringe, containing 4 mL of dichloroacetic acid (Cl₂CH—COOH) in dichloromethane (Cl₂CH₂), is made to pump the solution through the T-column and into the other syringe for several minutes. Once the 4 mL of dichloroacetic acid are pumped, the flow is reversed and the pumping continues toward the other (empty) syringe. This process is repeated at least three times. The reaction is deemed complete once yellow color does not come through the column. After the reaction is complete, the column is washed with 3 mL of dichloromethane at least two times.

Cleavage of 38-Mer from the T Column.

Cleavage of the 38-mer probe from the T column was achieved by in-column reaction with NH₄OH using the above procedure. The column is washed four times with 5 mL of NH₄OH with a residence time of fifteen minutes each time. The eluent is collected, and the last wash is left in the column overnight. The collected eluents are concentrated in a SpeedVac apparatus, and the amino-link 38-mer oligonucleotide undergoes deprotection for 12 hours at 55° C. Finally, the deprotected fractions of the amino-link 38-mer are combined and concentrated again in the SpeedVac, and their recovery is calculated by UV spectrophotometry. The final recovery was 130 nanomoles.

The dried 38-mer oligomer was dissolved in 2 mL of 0.1 M TEAA buffer, pH 7.5, and further purified by HPLC.

### HPLC Purification of the Amino-Link-Oligodeoxy Ribonucleotide

The aqueous mobile phase “A” for the HPLC purification was 0.1 M triethylammonium acetate, (TEAA, (CH₃—CH₂)₃—NH—OCO—CH₃), pH 7.0. Buffer A was prepared by dilution from a 1.0 M stock solution with HPLC grade water. The stock solution was prepared by adding slowly 57 mL (1.0 mol) of glacial acetic acid (CH₃—COOH) to 500 mL of an aqueous solution containing 139 mL (1.0 mol) of triethylamine (N(CH₂—CH₃) 3) at 5° C. degrees, while stirring. The solution was then made up to 1 L, and the pH adjusted to 7.0 with 1.0 M sodium hydroxide (NaOH). The organic phase “B” was 0.1 M TEAA in a 60% acetonitrile (CH₃—CN)/water mixture.

The HPLC system used consisted of a Hitachi L-6200 Intelligent pump (Hitachi Ltd., Tokyo, Japan); an L-3000 photo-diode array detector (Hitachi); a D-2500 Chromato Integrator (Hitachi), and an Epson Equity III computer connected to an Epson EX-800 printer (Seiko Epson Corp., Nagano, Japan).

Chromatographic separations were performed on a 0.46 cm×2.00 cm Supelco C-8 column (Sigma Aldrich, Burlington, MA) with 5 μm, 300 Å pore size particles. The oligomers were resolved by gradient elution. At time zero, 100% mobile phase “A” was pumped for 3 minutes, followed by a shallow linear gradient from 0% to 35% mobile phase “B” for 25 minutes. The column was then washed with a steeper linear gradient ramping from 35% to 90% mobile phase “B” for 26 minutes.

The 38-mer nucleotide probes (three synthesis, each at the 1 μM level) were separated using the procedure described above. The probes were collected on a redi-RAC fraction collector (Pharmacia LKB, Piscataway, New Jersey). Fractions were taken every 3 minutes; the desired product eluted on fractions 6 and 7. Samples were concentrated to dryness on a Savant SpeedVac (ThermoFisher Scientific, Inc., Rockville, MD) vacuum concentrator.

The amount of amino link in the probe was calculated by Fluram reaction with fluorescamine (Udenfriend et al., 1972, Science 178:871).

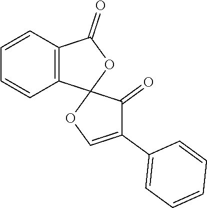 [SMILES: O=C1OC2(OC=C(c3ccccc3)C2=O)c2ccccc21]

### Example 5

### 38-Mer Aminolink Oligonucleotide Hybridization to M13mp19 DNA

A M13mp19 vector was linearized overnight using an ECO-RI enzyme (Sigma Chemical Co., St. Louis, MO). A quartz crystal containing 60 picomoles of 38-mer oligonucleotide was placed on a 20 mL scintillation vial and 1 mL of 1×SSC buffer was added. The scintillation vial was heated to 85° C. for 4 minutes. Linearized M13mp19 (2.1 μg, 10 μmol) was also placed in 6×SSC buffer and kept at 85° C. for 30 minutes.

Pre-incubation was followed by mixing of the linearized M13mp19 vector with the piezoelectric crystal at 85° C. for 2 minutes. The piezoelectric crystal and M13mp19 were then incubated together and allowed to hybridize overnight at 65° C. The reaction temperature was then lowered to 37° C. for 2 hours, followed for another temperature decrease to 4° C. At this point, the supernatant was tested, showing that 23.6% of the M13mp19 sequence had hybridized to the chip.

Hybridization of M13 Vector with 38-Mer Oligonucleotide.

The base-complementary alignment of the 38-mer oligonucleotide with the M13mp19 sequence is shown below:

Top strand: M13mp19 sequence (SEQ ID NO:2); bottom strand: oligonucleotide probe (SEQ ID NO: 1)

Xmal
Sacl EcoR1
5′-CCCCGGGTACCGAGCTCGAATTCACTGGCC
GTCGTTTTACAACGTCGTGACTGGGAAAACCCT
GG-′3
3′-TGACCGGCAGCAAAATGTTGCAGCACTGAC
CCTTTTG-NH₂-′5

### Example 6

### Loading of the 38-Mer Thio-Link Oligonucleotide to Activated Piezoelectric Crystal: Au—Hg— or Au—Hg-DTT

The washed piezoelectric crystals (Au—Hg or Au—Hg-DTT-SH) were placed into a 10-mL vial with 10 mL of 10 mM phosphate buffer, pH 7.0, and allowed to react with 300 nanomoles of the 38-mer thio-link oligonucleotide for at least 2 hours (or more, depending on target load).

The thio-link oligonucleotide load per crystal was determined either by dipping the piezoelectric crystal into a dithiothreitol (DTT) solution or by using NH₄OH as described above, and measuring the released amount of oligonucleotide by HPLC.

### Synthesis of 38-Mer DNA Thio-Linker

Thio-Link: DMT-S—(CH₂)₆—O—P—(OCNEt)N-(iPr)₂

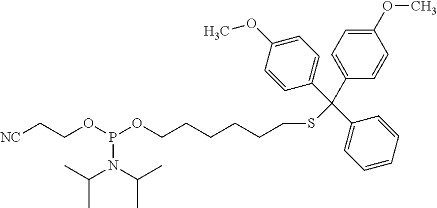 [SMILES: COc1ccc(C(SCCCCCCOP(OCCC#N)N(C(C)C)C(C)C)(c2ccccc2)c2ccc(OC)cc2)cc1]

DMT-SH-(link)-cyanoethyl phosphoramidite linker attachment to the 37-mer oligonucleotide sequence and cleavage of the trityl group.

150 mg of the linker compound was diluted with 4 mL of Biosearch Amidite diluent (Dry HCN Diluent); the linker compound is a viscous liquid that requires time to dissolve. The solution was transferred to the “X” reservoir of the Cyclone DNA synthesizer, and the sequence of the desired oligonucleotide entered with an “X” for the 5′ terminal base (38-residue-“SH”) or linker that needs to be incorporated. After priming the instrument, a normal oligonucleotide synthesis protocol was carried out, except that the synthesizer sequence was set so as to avoid removal of the final DMT. Cleavage of the MMT group was performed subsequently (see next paragraph).

Cleavage of the 38-Mer Oligonucleotide from the T Column

The cleavage of the 38-mer oligonucleotide from the control pore glass support and deprotection of the exocyclic amine groups is performed according to the usual protocol with NH₄OH for 1 hour at room temperature, followed for 5-10 hours at 55° C. The ammonium hydroxide is then removed by speed vacuum treatment. At this time, the oligomer is ready for storage.

### Thiol Deprotection Procedure

The dried, protected thio-38-mer oligomer was reconstituted into 0.1 M TEAA buffer, pH 7.5. Ten AU260 (absorbance units at 260 nm) of thio-linked oligonucleotide were reconstituted into 100 μL of TEAA. 15 μL of silver nitrate (AgNO₃) solution was added, vortexed, and allowed to react for 30 minutes at room temperature. Then, 20 μL of 1.0 M dithiothreitol (DTT) was added and vortexed for 15 minutes. The mixture turned yellow and, after centrifugation, the clear supernatant was collected. The pellet was washed with 100 μL of the TEAA buffer and centrifuged once more for 5 minutes.

The supernatant was again collected and combined with the first fraction. Ethyl acetate was added to the combined fractions to extract excess DTT. Once the excess DTT had been removed (top layer), the oligonucleotide solution is either: (1) kept under argon to prevent dimerization through disulfide formation; (2) dried; or (3) purified from the thio-link oligomer solution through reverse phase HPLC.

### Example 6

Reaction of the 38-Mer Amino-Link Oligonucleotide with a Bifunctional Probe

Reaction of the 38-mer amino-link oligonucleotide with a commercially available bifunctional reagent (e.g., sulfo-SMCC: (sulfosuccinimidyl-4-(N-maleimidomethyl)cyclohexane-1-carboxylate) crosslinker from ThermoFisher or Pierce, among other vendors) allows elongation of the linker and permits reaction with an Au—Hg piezoelectric crystal or Au—Hg-DTT crystal.

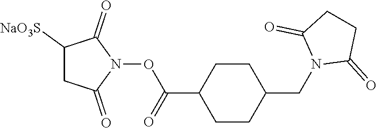

The derivatization is achieved by suspension of the amino-link oligomer in 300 μL of dimethyl formamide (DMF) and addition of the bifunctional crosslinking reagent (i.e., sulfo-SMCC, 300 μL in 0.1 M TEAA, pH 7.0-7.5), allowing the mixture to react overnight at 35° C.-37° C. The bifunctional reagent is added in excess (1:10 or 1:50 ratio; i.e.: 0.4 μM: 20 M). After removing the solvent, the residue is dried with anhydrous ether, the pellet resuspended in 0.1 M TEAA, pH 7.0, and washed again with anhydrous ether. A final resuspension in 0.1 M TEAA is carried out prior to separation and purification of the desired product by HPLC.

### Example 7

### Crystal Derivatization

A quartz crystal is used as a solid support. If the piezoelectric crystal has a gold electrode, the gold electrode is reacted with mercury (Hg) and then reacted with a thiol-linker DNA-probe. Unreacted sites are capped.

### Example 8

### Crystal Derivatization

A quartz crystal is used as a solid support. If the piezoelectric crystal has a gold electrode, the gold electrode is reacted with mercury (Hg), then reacted with a thiol link extender group (i.e., thiol-linker-maleimido), and then reacted with a thiolink DNA-probe. Unreacted sites are capped.

### Example 9

### Crystal Derivatization

A quartz crystal is used as a solid support. If the piezoelectric crystal has a gold electrode, the gold electrode is reacted with mercury (Hg), then reacted with a thiol link extender group (i.e. thio-link NHS (N-Hydroxy succinimide) and then reacted with an amino-link DNA-probe. Unreacted sites are capped.

### Example 10

### Crystal Derivatization

A quartz crystal is used as a solid support. A primary amine aminopropyl trimethoxy silane matrix is attached to the quartz crystal. The quartz crystal is then reacted with a crosslinking reagent such as EGS, sulfo-EGS and DSP. The crystals are then reacted with the corresponding thiol-link DNA probe or amino-link probe.

### Example 11

### Crystal Derivatization

Alumina with a pore size of 400 Å is used as a solid support. The approximate surface area per gram of aluminum metal is 10 m². An aluminum electrode may be used as a quartz crystal. The approximate surface area of the aluminum electrode will be 1.3×10⁽⁻⁵⁾ m². Ion exchange resins can be synthesized on the aluminum electrode. This protocol includes alumina modification with aminopropyl trimethoxy silane followed by branching with poly-L-Lysine. Lysines may be kept as such, or derivatized with a crosslinker. This protocol will allow to attach more than 500 picomoles of an amino-link probe or thiol-link-probe to the electrode. This is more than enough to obtain the proper signal once the system is scaled down, if needed for future in-house use.

### Example 12

### Crystal Sensor

A key component of a detection instrument is a quartz crystal sensor for frequency or phase shift measurements. An exemplary instrument utilizes a detection system based on a piezoelectric crystal resonator. Slight changes in mass, such as the addition of the complementary RNA chain to the DNA probe are readily measured by a corresponding change in resonance frequency. In the exemplary device, a mass change of 1 nanogram is equivalent to a frequency shift of 8 Hz on a crystal with a resonance frequency of 8,000,000 Hz. In this system/instrument the separation system and the detector are combined in the same compartment.

Viral or bacterial samples are lysed prior to testing, to release their nuclei nucleic acid. The released nucleic acid of interest is then captured by a nucleic acid probe (i.e., a DNA forward primer) immobilized on a piezoelectric crystal or stored in a buffer for further use. At this step, an internal DNA control can be added to control for degradation and amplification. The internal control is the reagent that monitors specimen stability, capture, elongation, amplification, and detection.

The quartz crystal can have attached an electrode of different compositions such as aluminum, silver, gold or gold amalgams. Measurements of an increase in mass can be determined using a suitable device, e.g., a microbalance or piezoelectric sensor. In one technique a change in the electrode mass is measured by a change in the frequency from the electrode to the crystal. For example, with the proposed technique, the DNA-probe coated crystal of the COVID-19 RNA with the proper sequence (complementary sequence) will diminish the chip frequency. Alternatively, it will be appreciated by those skilled in the art that a measurement may be made at the electrode of the crystal of phase shift or that a combination of frequency and phase shift measurements may be used for measurement of incorporation.

This technology can be used to detect any viral RNA using the proper specific probe or a universal probe. Additionally, this technology can also be used for viral or bacterial RNA or DNA detection if the bead is coated with a DNA primer (e.g., a second probe nucleic acid).

### Example 13

Attomole Detection of Viral RNA with DNA specific probes or Universal Probe coated Piezoelectric crystals.

A specific multiplexed diagnostic instrument comprises multiplexed piezoelectric surfaces (“n-crystals”) each coated with a different DNA probe, each probe directed against a different RNA virus (e.g., SARS-COV-2, Flu, SRV, Zika, etc.) placed in series, in parallel, or in other arranged form, e.g., pentameral, hexagonal, icosahedral (20) shape. Each face comprises an independent crystal, attached to an independent circuit.

After a viral RNA chain is hybridized to the corresponding crystal coated DNA-probe chain and elongated, excess reagents are washed away leaving the desired DNA/RNA hybrid attached to the piezoelectric crystal. The trapping can be made in a static mode or linear mode. In a static mode the RNA sample is delivered to the flow cell chamber in a minimum amount of volume with sufficient time for the complementary RNA chain to bind to the selected probe. Once hybridization occurs, excess solution is washed away. The hybridization time will depend on the length of the DNA-probe, and the flow cell or chamber volume.

After hybridization is complete the hybrid is elongated and, if enough RNA is present, a frequency shift will be detected immediately.

Signal amplification on the piezoelectric crystal is obtained immediately by adding a monoclonal antibody (MAB) coated bead with an antibody specific for DNA/RNA hybrids. The limit of detection (LOD) is below twenty picograms. A lower LOD can be achieved by adding larger MAB-coated-beads with increased mass or multiple smaller MAB-coated-beads.

The detector (quartz crystal) is placed in-line just outside of the reaction chamber. Any slight change in mass, such as the addition of the complementary RNA chain, is readily measured by a corresponding change in the resonance frequency.

### Example 14

### Quartz Crystal Signal Evaluation:

In one instance, the measurement of the antibody-coated bead attached to the hybrid probe attached to the crystal is interpreted as a positive signal once it exceeds a certain threshold. In another instance, the signal from the crystal is fed into a recording instrument to plot frequency changes and from the recorder the detected concentrations can be integrated by an integrator into digital data and input into a computer to determine accurate RNA or viral particle count.

Crystal evaluation is performed using an HP 4195A network analyzer or equivalent network analyzer as the measurement detection system. It is also advantageous to take the output measurements from the detection system and to display data on a recorder. In this example data is not available as a direct analog signal, but rather is in a digital form in IEEE-488 format, and a hardware and software interface is provided to adapt the digital output through a digital analog chip/board to a computer or to a cell phone. Further design of the flow cell allows for making crystal measurements in the crystal holder. Connections to the crystal used in the flow cell can be made through micro wire leads. This design along with a Teflon gasket or similar adaptation will afford a leakproof small volume flow cell.

### REFERENCES

1. Palva, A. (1986). Microbial diagnostics by nucleic acid hybridization. Annals of Clinical Research, 18 (5-6), 327-336.
2. Zwadyk, P., Cooksey, R. C., & Klevan, L. (1987). Nucleic acid probes in clinical microbiology. CRC Critical Reviews in Clinical Laboratory Sciences, 25 (1), 71-103.
3. Meinkoth, J., & Wahl, G. (1984). Hybridization of nucleic acids immobilized on solid supports. Analytical Biochemistry, 138 (2), 267-284.
4. Thein, S. L., and Wallace, R. B., Human Genetcs Diseases: A Practical Approach., (1986), David, K. E., Ed., IRL Press, Oxford, U. K. Chapter 3.
5. Li, P., Medon, P. P., Skingle, D. C., Lanser, J. A., & Symons, R. H. (1987). Enzyme-linked synthetic oligonucleotide probes: non-radioactive detection of enterotoxigenic Escherichia coli in faecal specimens. Nucleic Acids Research, 15 (13), 5275-5287.
6. Sanger, F., Nicklen, S., & Coulson, A. R. (1977). DNA sequencing with chain-terminating inhibitors. Proceedings of the National Academy of Sciences USA, 74 (12), 5463-5467.
7. Langer, P. R., Waldrop, A. A., & Ward, D. C. (1981). Enzymatic synthesis of biotin-labeled polynucleotides: novel nucleic acid affinity probes. Proceedings of the National Academy of Sciences USA, 78 (11), 6633-6637.
8. Brigati, D. J., Myerson, D., Leary, J. J., Spalholz, B., Travis, S. Z., Fong, C. K., . . . & Ward, D. C. (1983). Detection of viral genomes in cultured cells and paraffin-embedded tissue sections using biotin-labeled hybridization probes. Virology, 126 (1), 32-50.
9. Leary, J. J., Brigati, D. J., & Ward, D. C. (1983). Rapid and sensitive colorimetric method for visualizing biotin-labeled DNA probes hybridized to DNA or RNA immobilized on nitrocellulose: Bio-blots. Proceedings of the National Academy of Sciences USA, 80 (13), 4045-4049.
10. Stahl, S., Hultman, T., Olsson, A., Moks, T., & Uhlen, M. (1988). Solid phase DNA sequencing using the biotin-avidin system. Nucleic Acids Research, 16 (7), 3025-3038.
11. Tran, H. V., Piro, B., Reisberg, S., Duc, H. T., & Pham, M. C. (2013). Antibodies directed to RNA/DNA hybrids: an electrochemical immunosensor for microRNAs detection using graphene-composite electrodes. Analytical Chemistry, 85 (17), 8469-8474 . . .
12. Dang, T. T., & Morales, J. C. (2020). XRN2 Links RNA: DNA hybrid resolution to double strand break repair pathway choice. Cancers, 12 (7), 1821.
13. Cristini, A., Groh, M., Kristiansen, M. S., & Gromak, N. (2018). RNA/DNA hybrid interactome identifies DXH9 as a molecular player in transcriptional termination and R-loop-associated DNA damage. Cell Reports, 23 (6), 1891-1905.
14. Crossley, M. P., Bocek, M. J., Hamperl, S., Swigut, T., & Cimprich, K. A. (2020). qDRIP: a method to quantitatively assess RNA-DNA hybrid formation genome-wide. Nucleic Acids Research, 48 (14), e84-e84.
15. Mutharasan, Rajakkannu, Rijal, Kishan; Maraldo, Davis; and Campbell, Gossett, Augustus: Drexell University-PCT Patent Cooperation Treaty International Application: 29 Jan. 2009: WO 2009/014830 A1.
16. Nomura, T., & Okuhara, M. (1982). Frequency shifts of piezoelectric quartz crystals immersed in organic liquids. Analytica Chimica Acta, 142, 281-284.
17. Roederer, J. E., & Bastiaans, G. J. (1983). Microgravimetric immunoassay with piezoelectric crystals. Analytical Chemistry, 55 (14), 2333-2336.
18. Kanazawa, K. K., & Gordon II, J. G. (1985). The oscillation frequency of a quartz resonator in contact with liquid. Analytica Chimica Acta, 175, 99-105 . . .
19. Thompson, M., Arthur, C. L., & Dhaliwal, G. K. (1986). Liquid-phase piezoelectric and acoustic transmission studies of interfacial immunochemistry. Analytical Chemistry, 58 (6), 1206-1209.
20. Downs, M. E. (1991). Prospects for nucleic acid biosensors. Biochemical Society Transactions, 19 (1), 39-43.
21. Walton, P. W., Butler, M. E., & O'Flaherty, M. R. (1991). Piezoelectric-based biosensors. Biochemical Society Transactions, 19 (1), 44-48.
22. Hwang, C., Park, N., Kim, E. S., Kim, M., Kim, S. D., Park, S., . . . & Kim, J. H. (2021). Ultra-fast and recyclable DNA biosensor for point-of-care detection of SARS-COV-2 (COVID-19). Biosensors and Bioelectronics, 185, 113177.
23. Appella, D. H. (2009). Non-natural nucleic acids for synthetic biology. Current Opinion in Chemical Biology, 13 (5-6), 687-696.
24. Rasheed, P. A., & Sandhyarani, N. (2016). Quartz crystal microbalance genosensor for sequence specific detection of attomolar DNA targets. Analytica Chimica Acta, 905, 134-139.
25. Patolsky, Fernando, Willner, Itamar; Patent-International Patent Number-WO 02/20838 A2.-“Method and System for Detecting Nucleic Acids”.
26. Sessions, S. K. Genome Size. In Brenner's Encyclopedia of Genetics; Maloy, S., Hughes, K., Eds.; Elsevier: London, U K, 2013; Volume 3, pp. 301-305. ISBN 978-O-08-096156-9.
27. Subkhankulova, T., & Livesey, F. J. (2006). Comparative evaluation of linear and exponential amplification techniques for expression profiling at the single-cell level. Genome biology, 7 (3), 1-16.
28. Sanchez, J. A., Pierce, K. E., Rice, J. E., & Wangh, L. J. (2004). Linear-After-The-Exponential (LATE)-PCR: An advanced method of asymmetric PCR and its uses in quantitative real-time analysis. Proceedings of the National Academy of Sciences USA, 101 (7), 1933-1938.
29. Gibbons, H. R., & Aune, T. M. (2020). Immunoprecipitation of DNA: rNA hybrids using the S9. 6 antibody. RNA-Chromatin Interactions: Methods and Protocols, 195-207.
30. Braun, R. P., & Lee, J. S. (1987). Equilibrium binding parameters of an autoimmune monoclonal antibody specific for double-stranded DNA. Journal of Immunology, 139 (1), 175-179.
31. Li, Y., Miao, X., & Ling, L. (2015). Triplex DNA: A new platform for polymerase chain reaction-based biosensor. Scientific Reports, 5 (1), 13010.
32. Bagheri, E., Abnous, K., Alibolandi, M., Ramezani, M., & Taghdisi, S. M. (2018). Triple-helix molecular switch-based aptasensors and DNA sensors. Biosensors and Bioelectronics, 111, 1-9.
33 Applications of the Piezoelectric Quartz Crystal Microbalance. Methods and Phenomena 7. Lu, C. & Czauderna, A. W., eds., Elsevier, New York (1984).
34 Laibinis, P. E., Hickman, J. J., Wrighton, M. S., & Whitesides, G. M. (1989). Orthogonal self-assembled monolayers: alkanethiols on gold and alkane carboxylic acids on alumina. Science, 245 (4920), 845-847.
35. Campbell, G. A., & Mutharasan, R. (2006). Use of piezoelectric-excited millimeter-sized cantilever sensors to measure albumin interaction with self-assembled monolayers of alkanethiols having different functional headgroups. Analytical Chemistry, 78 (7), 2328-2334.
36 Introduction to Quartz Crystal Unit Design, by V. E. Bottom. Van Nostrand Reinhold Co., New York (1982).
37. Ngeh-Ngwainbi, J., Foley, P. H., Kuan, S. S., & Guilbault, G. G. (1986). Parathion antibodies on piezoelectric crystals. Journal of the American Chemical Society, 108 (18), 5444-5447.
38. Muramatsu, H., Dicks, J. M., Tamiya, E., & Karube, I. (1987). Piezoelectric crystal biosensor modified with protein A for determination of immunoglobulins. Analytical Chemistry, 59 (23), 2760-2763.
39. Guilbault, G. G., Luong, J. H., & Prusak-Sochaczewski, E. (1989). Immobilization methods for piezoelectric biosensors. Bio technology, 7 (4), 349-351.
40. Yang, J. Q., Yu, H. H., Jin, L., Yang, F. Z., Wu, D. Y., Zhan, D. P., & Tian, Z. Q. (2020). Insights into the effects of chloride ions on cyanide-free gold electrodeposition. Journal of The Electrochemical Society, 167 (10), 102514.
41. Udenfriend, S., Stein, S., Boehlen, P., Dairman, W., Leimgruber, W., & Weigele, M. (1972). Fluorescamine: a reagent for assay of amino acids, peptides, proteins, and primary amines in the picomole range. Science, 178 (4063), 871-872.
42. CRC Chemistry of Protein Conjugation and Crosslinking, by Shan S. Wong. CRC Press, Boca Raton, Florida (1993).
43. Bioconjugate Techniques, 2nd ed., by Hermanson, G. T. Academic Press, New York (2008).

The invention is further described by the following numbered paragraphs:

Paragraph 1. A method of detecting an target nucleic acid which comprises:
providing a probe nucleic acid immobilized to the surface of a sensor, wherein the probe nucleic acid comprises a sequence complementary to the target nucleic acid;
contacting the probe nucleic acid with the target nucleic acid to form a probe nucleic acid-target nucleic acid hybrid; and
determining whether the mass of the sensor is modulated, wherein a change in mass of the sensor indicates the presence of the target.

Paragraph 2. The method of paragraph 1, wherein the nucleic acid hybrid comprises a DNA-RNA hybrid, a DNA-DNA hybrid, or an RNA-RNA hybrid.

Paragraph 3. The method of paragraph 1, wherein the nucleic acid hybrid comprises a DNA-RNA duplex, a DNA-DNA duplex, or an RNA-RNA duplex.

Paragraph 4. The method of paragraph 1, wherein the method comprises elongation of one or more strands of the nucleic acid hybrid.

Paragraph 5. The method of paragraph 1, wherein the method comprises denaturing the nucleic acid hybrid and amplifying target nucleic acid.

Paragraph 6. The method of any one of paragraphs 1 to 4, wherein the method comprises contacting the nucleic acid hybrid with a molecular probe that specifically binds to the nucleic acid hybrid.

Paragraph 7. The method of paragraph 6, wherein the molecular probe comprises a monoclonal antibody.

Paragraph 8. The method of paragraph 6, wherein the molecular probe comprises a nucleic acid.

Paragraph 9. The method of any one of paragraphs 6 to 8, wherein the molecular probe is linked to a bead.

Paragraph 10. The method of paragraph 1, wherein the sensor comprises a piezoelectric material.

Paragraph 11. The method of paragraph 10, wherein the piezoelectric material comprises a quartz crystal.

Paragraph 12. The method of paragraph 10, wherein the sensor comprises a quartz crystal and an electrode comprising gold, silver, aluminum, or an amalgam.

Paragraph 13. The method of paragraph 1, wherein the target comprises a viral nucleic acid.

Paragraph 14. The method of paragraph 1, wherein the target comprises a bacterial nucleic acid.

Paragraph 15. The method of paragraph 1, wherein the change in mass of the sensor is determined from the frequency of the electrode.

Paragraph 16. The method of paragraph 1, wherein the change in mass of the sensor is determined from the phase angle of the electrode.

Paragraph 17. A system for identifying a target nucleic acid in a sample comprising:
(a) a surface capable of functioning as a transducer, the surface having bound thereto a probe nucleic acid complementary at the 3′ end to a segment of the target nucleic acid; and
(b) a polymerase capable of target nucleic acid-dependent addition of one or more nucleotides to the 3′ end of the probe nucleic acid to form an extended probe nucleic acid.

Paragraph 18. The system of paragraph 17, wherein the sensor comprises a piezoelectric sensor.

Paragraph 19. The system of paragraph 17, wherein the sensor comprises a quartz crystal and an electrode comprising gold, silver, aluminum, or an amalgam.

Paragraph 20. The system of paragraph 17, wherein the system comprises a molecular probe that specifically binds to a hybrid nucleic acid comprising the target nucleic acid and the extended probe nucleic acid.

Paragraph 21. The system of paragraph 20, wherein the molecular probe comprises an antibody or binding fragment thereof.

Paragraph 22. The system of paragraph 20, wherein the molecular probe is linked to a mass element.

Paragraph 23. The system of paragraph 20, wherein the molecular probe comprises a nucleic acid.

Paragraph 24. The system of paragraph 20, wherein the molecular probe comprises a nucleic acid linked to a mass element.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method of detecting a target nucleic acid which comprises:
providing a probe nucleic acid immobilized to the surface of a sensor, wherein the probe nucleic acid comprises a sequence complementary to the target nucleic acid;
contacting the probe nucleic acid with the target nucleic acid to form a probe nucleic acid-target nucleic acid hybrid; and
determining whether the mass of the sensor is modulated, wherein a change in mass of the sensor indicates the presence of the target.

2. The method of claim 1, wherein the nucleic acid hybrid comprises a DNA-RNA hybrid, a DNA-DNA hybrid, or an RNA-RNA hybrid.

3. The method of claim 1, wherein the nucleic acid hybrid comprises a DNA-RNA duplex, a DNA-DNA duplex, or an RNA-RNA duplex.

4. The method of claim 1, wherein the method comprises elongation of one or more strands of the nucleic acid hybrid.

5. The method of claim 1, wherein the method comprises denaturing the nucleic acid hybrid and amplifying target nucleic acid.

6. The method of claim 1, wherein the method comprises contacting the nucleic acid hybrid with a molecular probe that specifically binds to the nucleic acid hybrid.

7. The method of claim 6, wherein the molecular probe comprises a monoclonal antibody.

8. The method of claim 6, wherein the molecular probe comprises a nucleic acid.

9. The method of claim 6, wherein the molecular probe is linked to a bead.

10. The method of claim 1, wherein the sensor comprises a piezoelectric material.

11. The method of claim 10, wherein the piezoelectric material comprises a quartz crystal.

12. The method of claim 10, wherein the sensor comprises a quartz crystal and an electrode comprising gold, silver, aluminum, or an amalgam.

13. The method of claim 1, wherein the target comprises a viral nucleic acid.

14. The method of claim 1, wherein the target comprises a bacterial nucleic acid.

15. The method of claim 1, wherein the change in mass of the sensor is determined from the frequency of the electrode.

16. The method of claim 1, wherein the change in mass of the sensor is determined from the phase angle of the electrode.

17. A system for identifying a target nucleic acid in a sample comprising:
(a) a surface capable of functioning as a transducer, the surface having bound thereto a probe nucleic acid complementary at the 3′ end to a segment of the target nucleic acid; and
(b) a polymerase capable of target nucleic acid-dependent addition of one or more nucleotides to the 3′ end of the probe nucleic acid to form an extended probe nucleic acid.

18. The system of claim 17, wherein the sensor comprises a piezoelectric sensor.

19. The system of claim 17, wherein the sensor comprises a quartz crystal and an electrode comprising gold, silver, aluminum, or an amalgam.

20. The system of claim 17, wherein the system comprises a molecular probe that specifically binds to a hybrid nucleic acid comprising the target nucleic acid and the extended probe nucleic acid.

21. The system of claim 20, wherein the molecular probe comprises an antibody or binding fragment thereof.

22. The system of claim 20, wherein the molecular probe is linked to a mass element.

23. The system of claim 20, wherein the molecular probe comprises a nucleic acid.

24. The system of claim 20, wherein the molecular probe comprises a nucleic acid linked to a mass element.
